(19) United States
(12) Patent Application Publication
(10) Pub. No.: US 2011/0144383 A1
(43) Pub. Date: Jun. 16, 2011

(54) PROCESS FOR PREPARING (S)-3-(AMINOMETHYL)-5-METHYLHEXANOIC ACID

(75) Inventors: Ataharoddin Khala, Jinnaram Mandal (IN); Venkata Srinivas Rao Potla, Jinnaram Mandal (IN); Govind Singh Rawat, Jinnaram Mandal (IN); Babu Rao Konudula, Jinnaram Mandal (IN); Yogendra Kumar Chauhan, Jinnaram Mandal (IN); Debashish Datta, Jinnaram Mandal (IN)

(73) Assignee: MATRIX LABORATORIES LIMITED, Secunderabad, Andhra Pradesh (IN)

(21) Appl. No.: 12/918,046

(22) PCT Filed: Feb. 18, 2009
(86) PCT No.: PCT/IN2009/000111

§ 371 Date: Oct. 5, 2010

(30) Foreign Application Priority Data
Feb. 18, 2008 (IN) 410/CHE/2008

(51) Int. Cl. C07C 227/34

(52) U.S. Cl. 562/401

(57) ABSTRACT

Disclosed herein is a process for the preparing (S)-3-(aminomethyl)-5-methylhexanoic acid by optical resolution of (±)-3-(aminomethyl)-5-methylhexanoic acid and a resolving agent employing a suitable solvent.

### FIELD OF THE INVENTION

This invention, in general relates to a process for preparing (S)-3-(aminomethyl)-5-methylhexanoic acid. In particular, the present invention provides an improved resolution process for preparing (S)-3-(aminomethyl)-5-methylhexanoic acid ((S)-Pregabalin) from (±)-3-(aminomethyl)-5-methylhexanoic acid and a resolving agent employing a suitable solvent.

### BACKGROUND OF THE INVENTION

(S)-(+)-3-(aminomethyl)-5-methylhexanoic acid [(S)-Pregabalin], a compound having the chemical structure of formula (I),

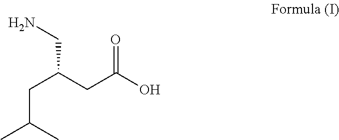

is a γ-amino butyric acid (GABA) analogue. (S)-3-(aminomethyl)-5-methylhexanoic acid has been found to activate GAD (L-glutamic acid decarboxylase). It is a CNS-active compound and has a dose dependent protective effect on-seizure. (S)-3-(aminomethyl)-5-methylhexanoic acid is useful in anticonvulsant therapy, due to its activation of GAD, promoting the production of GABA, one of the brain's major inhibitory neurotransmitters, which is released at 30 percent of the brains synapses.

The pharmacological activity of pregabalin is primarily attributable to the S-enantiomer and thus, several methods have been developed to prepare the S-enantiomer of pregabalin substantially free of the R-enantiomer.

Preparation of (S)-Pregabalin from the resolution of isobutyl-GABA is disclosed in U.S. Pat. No. 5,637,767, by reacting (±)-3-(aminomethyl)-5-methylhexanoic acid with (S)-mandelic acid in water, an alcohol or a mixture of water and an alcohol; allowing a precipitate to form; introducing the precipitate into a polar aprotic solvent or a mixture of polar aprotic solvent and water to form a slurry; and collecting the solid from the slurry.

In addition (S)-3-(aminomethyl)-5-methylhexanoic acid can be prepared, as disclosed in U.S. Pat. No. 5,616,793, and in Drugs of The Future, 24 (8), 862-870 (1999) by obtaining the intermediate, 3-(carbamoylmethyl)-5-methylhexanoic acid (“CMH”), which is then optically resolved to give R-CMH, which is then converted to (S)-Pregabalin, as described in the following scheme:

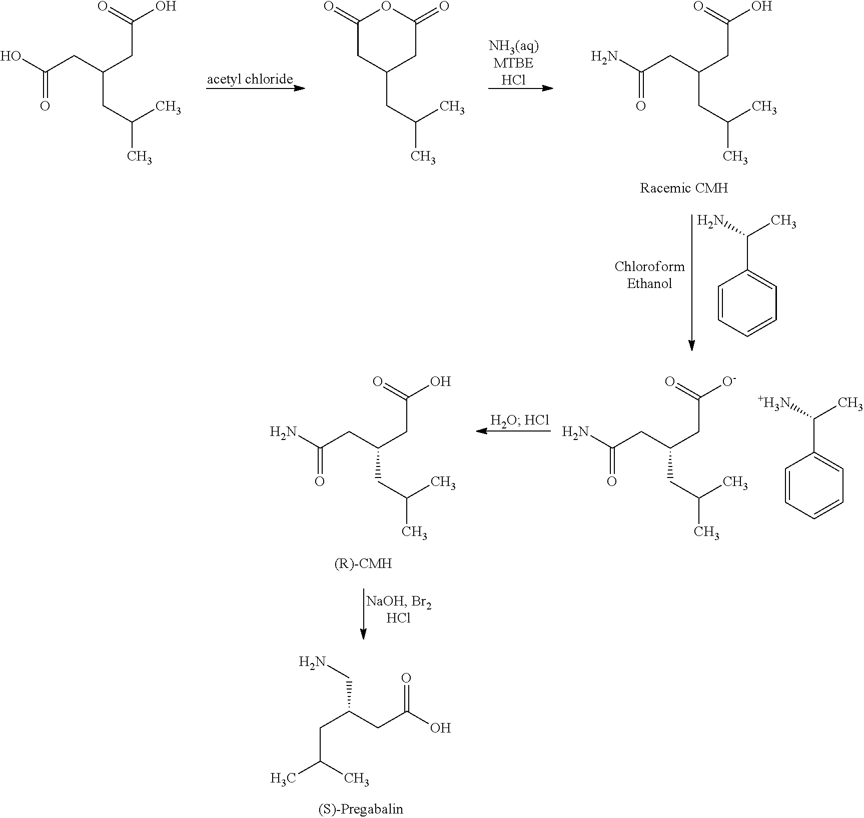

The disadvantage of the prior art process is the difficultly of handling bromine, which is hazardous chemical. So there is a need for an improved process, which will avoid the usage of hazardous chemicals and is industrially viable with product of high purity and yield.

Accordingly, the present invention provides an improved process for producing S-stereoisomer of 3-isobutyl GABA.

### OBJECT AND SUMMARY OF THE INVENTION

It is an object of the present invention to provide an improved resolution process for preparing (S)-3-(aminomethyl)-5-methylhexanoic acid ((S)-Pregabalin) from (±)-3-(aminomethyl)-5-methylhexanoic acid.

It is another object of the present invention is to provide an improved resolution process for preparing (S)-3-(aminomethyl)-5-methylhexanoic acid ((S)-Pregabalin), wherein said process provides the product in high yield and purity employing a resolving agent in presence of a suitable solvent.

The above and other objects of the present invention are further attained and supported by the following embodiments described herein. However, the scope of the invention is not restricted to the described embodiments herein after.

In accordance with one preferred embodiment of the present invention, there is provided a resolution process for preparing (S)-3-(aminomethyl)-5-methylhexanoic acid from (±)-3-(aminomethyl)-5-methylhexanoic acid, which comprises combining the racemic 3-(aminomethyl)-5-methylhexanoic acid with (S)-mandelic acid in water, an alcohol, ketone or mixture thereof, allowing a precipitate to form, introducing the precipitate into a polar protic or non polar solvent to form a slurry optionally in presence of a organic base and collecting the resultant solid from the slurry.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The present invention describes the resolution process for the preparation (S)-3-(aminomethyl)-5-methylhexanoic acid or its salt thereof from (±)-3-(aminomethyl)-5-methylhexanoic acid or its salt thereof.

According to the present invention, there is provided a resolution process for the preparation of (S)-3-(aminomethyl)-5-methylhexanoic acid or its salt thereof from (±)-3-(aminomethyl)-5-methylhexanoic acid or its salt thereof, which comprises combining the racemic 3 -(aminomethyl)-5-methylhexanoic acid with (S)-mandelic acid in water, an alcohol, ketone or mixture thereof, allowing a precipitate to form, introducing the precipitate into a polar protic or non polar solvent to form a slurry optionally in presence of base and isolating the solid from the slurry.

In one of the embodiment of the present invention, (±)-3-(aminomethyl)-5-methylhexanoic acid and S-(+) Mandelic acid are combined in a mixture of water and alcohol to get S-(+)-3-(Amino methyl)-hexanoic acid -S-(+) Mandelic acid salt, wherein the alcohol is preferably isopropylalcohol.

In one of the embodiment of the present invention, (±)-3-(aminomethyl)-5-methylhexanoic acid and S-(+) Mandelic acid are combined in a mixture of water and ketone to get S-(+)-3-(Amino methyl)-hexanoic acid -S-(+) Mandelic acid salt, wherein ketone is preferably acetone.

The polar protic solvent employed to form slurry according to the present invention is selected from methanol, ethanol and isopropanol, preferably methanol.

The non polar solvent employed to form slurry according to the present invention is selected from hexane, cyclohexane and heptane.

The organic base according to the present invention is selected from triethylamine, diisopropylamine, N, N-Diisopropylethylamine, di-methylamine, preferably triethylamine.

The synthetic scheme of (S)-Pregabalin is depicted in the following synthetic scheme:

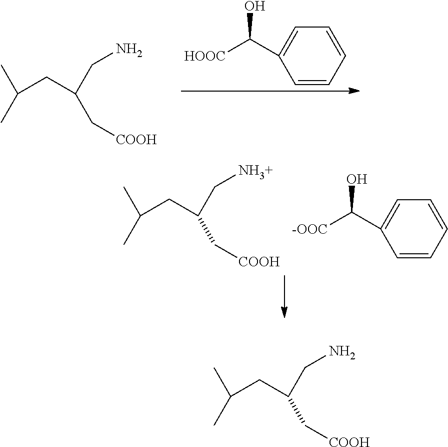

According to the present invention, (S)-3-(aminomethyl)-5-methylhexanoic acid or its salt thereof is prepared by treating the racemic 3-(aminomethyl)-5-methylhexanoic acid with (S)-mandelic acid in water, alcohol, ketone or mixture thereof, preferably water and alcohol mixture more preferably isopropylalcohol and water mixture and allowing the precipitate to form. The obtained precipitate is further treated with polar protic or non polar solvent optionally in presence of base to get the (S)-3-(aminomethyl)-5-methylhexanoic acid. Polar protic solvent is selected from methanol, ethanol, isopropanol, more preferably methanol and base is organic base selected from triethylamine, diisopropylamine, N, N-Diisopropylethylamine, dimethylamine more preferably triethylamine.

As per the present invention (S)-Pregabalin is prepared with high purity and high yield.

The following examples are provided to illustrate the process of the present invention. They, are however, not intended to limiting the scope of the present invention in any way and several variants of these examples would be evident to person ordinarily skilled in the art.

### EXAMPLES

### Example-1

### Preparation of 3-Nitromethyl-5-Methyl Hexanoic Acid Benzyl Ester

Benzyl bromoacetate (100 g) and Triethyl phosphite (79.79 g) were taken in a round-bottomed flask and the reaction mass was heated for ˜60 minutes at 65-70 ° C., after the completion of reaction, reaction mass was further heated to 70-85 ° C. and later cooled to ambient temperature. To this tetrahydrofuran was added, followed by addition of 1,8-Diazabicyclo[5.4.0]undec-7-ene (80 g) and Isovaleraldehyde (47.04 g). Reaction mass was maintained at room temperature. After completion of the reaction resulting solution was acidified with dilute hydrochloric acid and extracted with ethyl acetate and ethyl acetate layer was concentrated. To concentrated residue tetrahydrofuran was added followed by 1,8-Diazabicyclo[5.4.0]undec-7-ene (76.8 g) and Nitromethane (140 g). After completion of the reaction resulting solution was acidified with dil hydrochloric acid and extracted with ethyl acetate to obtain 3-Nitromethyl-5-methyl hexanoic acid benzyl ester

### Example-2

### Preparation of Racemic Pregabalin

Methanol (1500 ml) and 3-Nitromethyl-5-methyl hexanoic acid benzyl ester (100 g) were taken in an autoclave. To this 5% Pd/C (30 g) was added. The mixture was hydrogenated under 4-5 kg/cm² and the reaction mass was filtered and washed with methanol followed by concentration. To the concentrated residue, mixture of isopropylalcohol and water was added to get the racemic Pregabalin.

### Example-3

### Preparation of S-(+)-3-(Amino Methyl)-Hexanoic Acid -S-(+) Mandelic Acid Salt

Racemic Pregabalin (100 g) was taken in a mixture of isopropyl alcohol, water and heated to 50-55 ° C. To the reaction mixture S-(+)-Mandelic Acid (132 g) was added and reaction mass was cooled to 25-30 ° C. and filtered. To the filtered reaction mass, mixture of isopropyl alcohol and water was added and heated to get the clear solution, then second lot of S-(+)-Mandelic Acid (19.5 g) was added and slowly cooled. The obtained solid was filleted, washed with isopropyl alcohol and dried to get the Mandelic salt.

### Example-4

### Preparation of Crude S-(+)-3-(Amino Methyl)-5-Methyl Hexanoic Acid

S-(+)-3-(Amino methyl)-hexanoic acid S-(+) Mandelic acid salt, was taken in methanol (800 ml) and gently heated to obtain clear solution. Methanolic triethylamine (30 gm triethylamine in 200 ml methanol) was slowly added to the clear solution and cooled to 10-15 ° C. The obtained solid was filtered and dried to get Crude S-(+)-3-(Amino methyl)-5-methyl hexanoic acid.

### Example-5

### Preparation of S-(+)-3-(Amino Methyl)-5-Methyl Hexanoic Acid

Crude S-(+)-3-(Amino methyl)-5-methyl hexanoic acid (100 g) was taken in a mixture of isopropyl alcohol (1500 ml) and water (500 ml) and heated to 65-75 ° C. to obtain clear solution. The solution was cooled to 0-5 ° C. The obtained solid was filtered and dried to yield pure S-(+)-3-(Amino methyl)-5-methyl hexanoic acid.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

We claim:

1. A process for preparing S-(+)-3-(Amino methyl)-5-methyl hexanoic acid by optical resolution of (±)-3-(aminomethyl)-5-methylhexanoic acid, the process comprising:
(a) combining (±)-3-(aminomethyl)-5-methylhexanoic acid, a solvent selected from alcohol, ketone and water or mixture thereof and S-(+) Mandelic acid as a resolving agent to obtain a reaction mixture;
(b) allowing the resultant reaction mixture to form a precipitate;
(c) combining the resultant precipitate with a solvent selected from polar protic or non polar solvent or mixture thereof, optionally in presence of a base to form a slurry; and
(d) isolating the resultant pure solid S-(+)-3-(Amino methyl)-5-methyl hexanoic acid from slurry.

2. The process according to claim 1, wherein the solvent used in step (a) is a mixture of water and alcohol.

3. The process according to claim 2, wherein the alcohol is isopropylalcohol.

4. The process according to claim 1, wherein the solvent used in step (a) is a mixture of water and ketone.

5. The process according to claim 1, wherein the polar protic solvent is selected from methanol, ethanol, isopropanol or mixture thereof.

6. The process according to claim 1, wherein the non polar solvent is selected from hexane, cyclohexane and heptane.

7. The process according to claim 1, wherein the polar protic solvent is methanol.

8. The process according to claim 1, wherein the base is organic base.

9. The process according to claim 8, wherein organic base is selected from triethylamine, diisopropylamine, N, N-Diisopropylethylamine and dimethylamine.
